(19) United States
(12) Patent Application Publication
(10) Pub. No.: US 2009/0214419 A1
(43) Pub. Date: Aug. 27, 2009

(54) SELF-ASSEMBLED BIODEGRADABLE POLYMERSOMES

(75) Inventors: Michael J. Therien, Philadelphia, PA (US); Daniel A. Hammer, Villanova, PA (US); Paiman Peter Ghoroghchian, Downingtown, PA (US); Guizhi Li, Rochester, NY (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: The Trustees of the University of Pennsylvania, Philadelphia, PA (US)

(21) Appl. No.: 12/088,343

(22) PCT Filed: Sep. 28, 2006
(86) PCT No.: PCT/US06/38189

§ 371 Date: Dec. 3, 2008

Related U.S. Application Data
(60) Provisional application No. 60/721,163, filed on Sep. 28, 2005.
(60) Provisional application No. 60/761,322, filed on Jan. 23, 2006.

(51) Int. Cl. A61K 51/12; C08G 63/08; A61K 38/02; A61K 31/70; A61K 33/00; A61K 31/00; A61K 49/18; A61K 49/04; B05D 3/00

(52) U.S. Cl. 424/1.21; 525/450; 514/2; 514/23; 424/600; 514/1; 424/9.3; 424/9.4; 427/2.14

(57) ABSTRACT

The invention concerns a block copolymer of polyethylene oxide and polycaprolactone, the polyethylene oxide having a number average molecular weight from about 2.0 to about 3.8 kD, the block copolymer having a fraction of polyethylene oxide of from about 11.8 to 18.8 percent by weight. The invention also concerns polymersomes made from such copolymers and to methods of making the polymersomes.

### RELATED APPLICATIONS

This application claims benefit to U.S. Provisional Application No. 60/761,322, filed Jan. 23, 2006, and U.S. Provisional Application No. 60/721,163, filed Sep. 28, 2005.

### FIELD OF THE INVENTION

The invention concerns biodegradable polymersomes, more particularly, polymersomes made of poly(ethyleneoxide)-b-polycaprolactone diblock copolymers.

### BACKGROUND OF THE INVENTION

Polymersomes, 50 nm-50 μm diameter vesicles formed from amphiphilic block copolymers, have attracted much attention due to their superior mechanical stabilities and unique chemical properties when compared to conventional lipid-based vesicles (liposomes) and micelles. See, generally, Discher, D. E.; Eisenberg, A. Science, 2002, 297, 967-973; Discher, B. M.; Won, Y. Y.; Ege, D. S.; Lee, J. C. M.; Bates, F. S.; Discher, D. E.; Hammer, D. A. Science 1999, 284, 1143-1146; Lee, J. C. M.; Bermudez, H.; Discher, B. M.; Sheehan, M. A.; Won, Y. Y.; Bates, F. S.; Discher, D. E. Biotechnol. Bioeng. 2001, 73, 135-145, Bermudez, H.; Brannan, A. K.; Hammer, D. A.; Bates, F. S.; Discher, D. E., Macromolecules 2002, 35, 8203-8208, and Ghoroghchian, P. P.; Frail, P. R.; Susumu, K.; Blessington, D.; Brannan, A. K.; Bates, F. S.; Chance, B.; Hammer, D. A.; Therien, M. J. Proceedings of the National Academy of Sciences of the United States of America (PNAS), 2005, 102, 2922-2927. Polymer vesicles have further proven capable of not only entrapping water-soluble hydrophilic compounds (drugs, vitamins, fluorophores, etc.) inside of their aqueous cavities but also hydrophobic molecules within their thick lamellar membranes. Moreover, the size, membrane thickness, and stabilities of these synthetic vesicles can be rationally tuned by selecting block copolymer chemical structure, number-average molecular weight, hydrophilic to hydrophobic volume fraction, and via various preparation methods. Polymersomes thus have many attractive characteristics that lend to their potential application in medical imaging, drug delivery, and cosmetic devices. See, Discher, D. E.; Eisenberg, A. Science, 2002, 297, 967-973, Frail, P. R.; Susumu, K.; Blessington, D.; Brannan, A. K.; Bates, F. S.; Chance, B.; Hammer, D. A.; Therien, M. J. Proceedings of the National Academy of Sciences of the United States of America (PNAS), 2005, 102, 2922-2927 and Meng F.; Engbers, G. H. M.; Feijen J. Journal of Controlled Release, 2005, 101, 187-198.

To date, polymersomes have been formed from a number of different amphiphilic block copolymers, including poly(ethylene oxide)-b-polybutadiene (PEO-b-PBD), poly(ethylene oxide)-b-polyethylethylene (PEO-b-PEE), polystyrene-b-poly(ethylene oxide) (PS-b-PEO), poly(ethylene oxide)-b-poly(propylene oxide)-b-poly(ethylene oxide) (PEO-b-PPO-b-PEO) triblock copolymer, polystyrene-b-poly(acrylic acid) (PS-b-PAA), poly(2-methyloxazoline)-b-poly(dimethylsiloxane)-b-poly(2-methyloxazoline) (PMOXA-b-PDMS-b-PMOXA), etc. None of these formulations, however, yields self-assembled fully-biodegradable polymersomes necessary for human in vivo applications. Feijen et al reported that biodegradable polymersomes could be prepared from amphiphilic biodegradable diblock copolymers of PEO and aliphatic polyesters/polycarbonates by using an organic co-solvent/water injection/extraction system. See, Meng F.; Hiemstra, C.; Engbers, G. H. M.; Feijen J. Macromolecules, 2003, 36, 3004-3006. In comparison with other polymersome preparation methods based on self-assembly, i.e. film hydration, bulk hydration or electroformation, the major drawback of this system is that the organic co-solvent must be completely removed from the aqueous polymersome suspension post-assembly. In addition to the extra time and cost associated with such processing, any residual organic solvent would be highly toxic within the human body.

There is a need in the art for fully biodegradable polymersomes that are easily assembled and do not have the drawbacks of a system assembled with the use of an organic/water co-solvent/extraction system.

### SUMMARY

In some embodiments, the invention concerns vesicles or polymersomes made from block copolymers of polyethylene oxide and polycaprolactone. The polyethylene oxide can have a number average molecular weight from about 2.0 to about 3.8 kD. The block copolymer may have a fraction of polyethylene oxide from about 11 to 20 percent by weight. In some embodiments, the fraction of polyethylene oxide is from about 12 to 19 percent by weight. In other embodiments, the fraction is from 11.8 to 18.8 percent by weight.

In some aspects, the invention concerns methods for preparing the aforementioned polymersomes. In certain preferred embodiments, the polymersomes are capable of self-assembly.

### BRIEF DESCRIPTION OF THE DRAWINGS

FIG. 1 presents a representative ¹H-NMR spectrum of PEO-B-PCL diblock copolymer.

FIG. 2 shows confocal laser fluorescence micrographs (λex=488 nm) of PEO(2k)-b-PCL(12k)-based polymersomes containing membrane-encapsulated Nile Red and aqueous-internalized calcein dyes.

FIG. 3 presents a confocal laser fluorescence micrograph of polymersomes comprised of a mixture of PEO(2k)-b-(9.5k)/PEO(2k)-b-(12k)/PEO(2k)-b-PCL(15k), formed by aqueous hydration of a thin-film of the polymers deposited in 1:1:1 molar ratio on Teflon®.

FIG. 4 shows a cryogenic transmission electron micrograph of PEO(2k)-b-PCL(12k)-based vesicles in DI water (5 mg/mL). The membrane core-thickness of the vesicles is 22.5+/−2.3 nm.

FIG. 5 shows microspheres ((a) optical micrograph and (b) confocal fluorescence micrograph) derived from organic co-solvent extraction of PEO(5k)-b-PCL(52k) in aqueous solution.

FIG. 6 presents differential scanning calorimetry to elucidate the thermal transitions of PEO(2k)-b-PCL(12k).

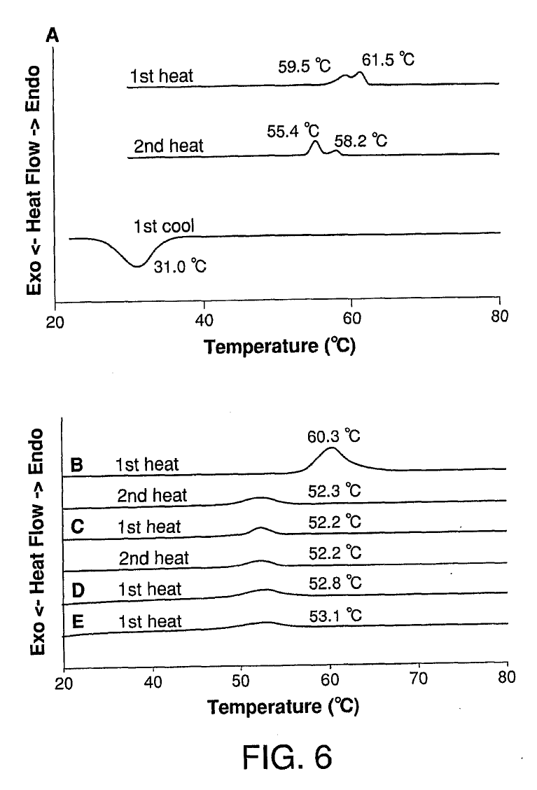

FIG. 7 illustrates methods of preparation of polymersomes.

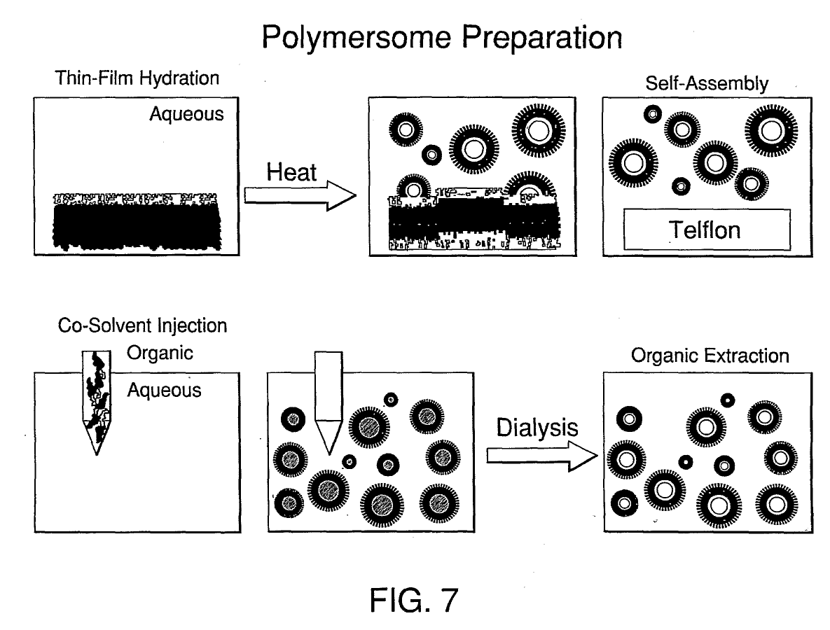

FIG. 8 shows in situ DOX release in various physiological conditions (pH 5.5 and 7.4; T=37° C.).

FIG. 9 shows a cumulative histogram of the size distribution of PEO(2k)-PCL(12k)-based polymersomes as obtained via dynamic light scattering (DLS) at 25° C.

FIG. 10 shows H¹-NMR spectra of PEO(2K)-B-PCL(12K) diblock copolymer (A) before and (B) after generation of 200 nm diameter polymersomes via thin-film self assembly (65° C. for 1 hr) and subsequent sizing via 5 cycles of freeze-thaw extraction and extrusion.

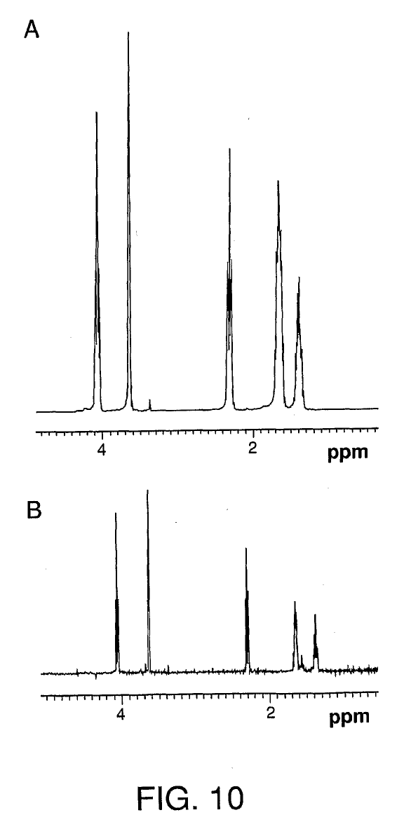

### DETAILED DESCRIPTION OF ILLUSTRATIVE EMBODIMENTS

In some embodiments, the invention concerns vesicles that are constructed partially or entirely of a block copolymer of polyethylene oxide and polycaprolactone. The polyethylene oxide can have a number average molecular weight from about 2.0 to about 3.8 kD. The block copolymer can have a fraction of polyethylene oxide from about 11 to about 20 percent by weight. In certain embodiments, the block copolymer can have additional monomeric units or blocks. These blocks can be additional polyethylene oxide or polycaprolactone blocks or they can be of a different material.

In some embodiments, the polymersome contains a hydrophobic polycaprolactone, polylacticde, polyglycolide, or polymethylene carbonate polymer block used in combination with a corresponding polyethyleneoxide polymer block. In certain embodiments, the polymersome is based upon an amphilphilic random copolymer consisting of a discrete polyethylene oxide block and a random hydrophobic polymer block in which there exists an oligocaprolactone component, and hydrophobic polylacticde, polyglycolide, or polymethylene carbonate oligomers.

The amphiphilic co-polymer can comprise polymers made by free radical initiation, anionic polymerization, peptide synthesis, or ribosomal synthesis using transfer RNA.

In some embodiments, the vesicle has a fraction of polyethylene oxide of from about 12 to about 19 percent by weight. In other embodiments, the vesicle has a fraction of polyethylene oxide of from about the 11.8 to 18.8 percent by weight.

It should be noted that one can use poly(ethylene glycol) (PEG) as the hydrophilic block in the compositions described herein. PEG is known to have similar properties to polyethylene oxide (PEO). As such, poly(ethylene glycol)-polycaprolactone diblocks should function in the same manner as polyethylene oxide-polycaprolactone compositions. Additional compositions that are within the scope of the invention are mixtures of two or more PEO, polypropylene oxide (PPO), and PEG.

The number average molecular weight of the polyeaprolactone is from about 9 to about 23 kD. In some embodiments, the number average molecular weight of the polycarporlactone is from about 9.5 to about 22.2 kD. Mn is determined in our studies by GPC and NMR. Such techniques are standard methods known to those skilled in the art. See, for example, Polymer Handbook, Volumes 1-2, Fourth Edition, J. Brandrup (Editor), Edmund H. Immergut (Editor), Eric A. Grulke, Akihiro Abe, Daniel R. Bloch; ISBN: 0-471-47936-5; and Block Copolymers: Synthetic Strategies, Physical Properties, and Applications, Nikos Hadjichristidis, Stergios Pispas, George Floudas, ISBN: 0-471-39436-X.

In one preferred embodiment, the molecular weight of the polyethylene oxide is about 2 kD and the molecular weight of the polycaprolactone is about 12 kD.

In certain preferred embodiments, the vesicle is bioresorbable. The term “bioresorbable” refers to a molecule, when degraded by chemical reactions, leads to substituents which can be used by biological cells as building blocks for the synthesis of other chemical species, or can be excreted as waste.

Polyethyleneoxide is a hydrophilic block which imparts to the vesicle's surface biocompatibility and prolonged blood circulation times. Polycaprolactone (PCL) constitutes the vesicles' hydrophobic membrane portion. PCL is degraded by hydrolysis of its ester linkages in physiological conditions (such as in the human body) and has therefore received a great deal of attention for use as an implantable biomaterial in drug delivery devices, bioresorbable sutures, adhesion barriers, and as a scaffold for injury repair via tissue engineering. Compared to other biodegradable aliphatic polyesters, PCL has several advantageous properties including: 1) high permeability to small drug molecules; 2) maintenance of a neutral pH environment upon degradation; 3) facility in forming blends with other polymers; and 4) suitability for long term delivery afforded by slow erosion kinetics as compared to polylactide (PLA), polyglycolide (PGA), and polylactic-co-glycolic acid (PLGA). Utilization of PCL as the hydrophobic block in our formulations insures that the resultant polymersomes will have safe and complete in vivo degradation.

Amphiphilic polyethyleneoxide-b-polycaprolactone can be generated via ring-opening polymerization of cyclic ε-caprolactone (CL) in the presence of stannous(II) octoate (SnOct) and monocyano- or monomethoxy-poly(ethyleneoxide) (PEO, 0.75 k, 1.1 k, 1.5 k, 2 k, 5 k, 5.5 k, 5.8 k; Polymer Source, Dorval, Canada). See, Bogdanov, B.; Vidts, A.; Van Den Bulcke, A.; Verbeeck, R.; Schacht, E. Polymer 1998, 39, (8-9), 1631-1636.

The compositions of the instant invention allow the generation of self-assembled vesicles comprised entirely of an amphiphilic diblock copolymer of polyethyleneoxide (PEO) and polycaprolactone (PCL), two previously FDA-approved polymers. Unlike degradable polymersomes formed from blending “bio-inert” and hydrolysable components, PEO-b-PCL-based vesicles should be fully bioresorbable, leaving no potentially toxic byproducts upon their degradation. Moreover, unlike published reports of other degradable (e.g. peptide, polyester, or polyanhydride-based) vesicles, these bioresorbable polymersomes are formed spontaneously through self-assembly of pure PEO-b-PCL diblock copolymer.

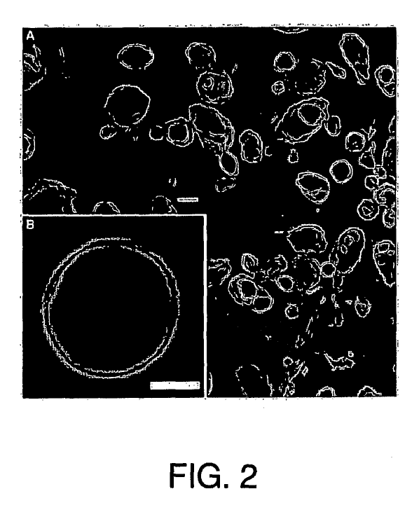

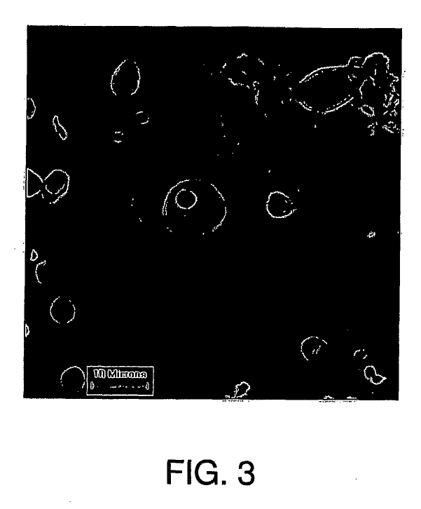

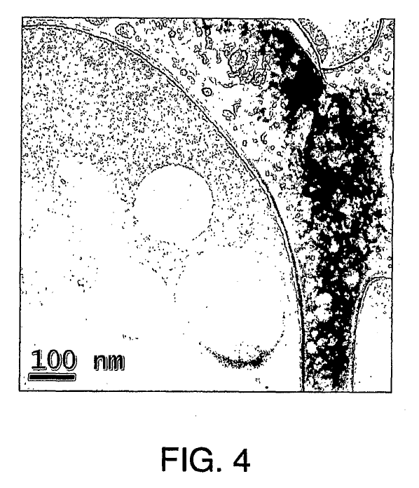

The size distributions of the polymersomes can be controlled with standard techniques such as sonication, freeze/thaw extraction, and extrusion above a Tm=60° C., to yield mono-dispersed vesicle diameters ranging anywhere from tens of microns (confocal micrographs, FIGS. 2 and 3) to hundred nanometer-sized structures (cryo-TEM micrograph, FIG. 4) useful for in vivo applications. See, Moghimi, S. M.; Hunter, A. C.; Murray, J. C. Pharmacol. Rev. 2001, 53, (2), 283-318 and Ghoroghchian, P. P.; Frail, P. R.; Susumu, K.; Blessington, D.; Brannan, A. K.; Bates, F. S.; Chance, B.; Hammer, D. A.; Therien, M. J. Proc. Natl. Acad. Sci. USA 2005, 102, (8), 2922-2927.

Particularly preferred are PEO(2k-3.8k)-b-PCL(9.5-22.2k) diblock copolymers (wt, f_{PEO} ranging from 11.8-18.8%) which can self-assemble into biodegradable polymersomes. The molecular weight distribution of these polymers does not appear to be important (i.e. PDI does not need≦1.2).

While it is believed that all previously known vesicle-generating, self-assembled, amphiphilic diblock polymers possess a hydrophilic volume fraction of 0.3-0.4 (see, Discher, D. E.; Eisenberg, A. Science 2002, 297, (5583), 967-973 and Bermudez, H.; Brannan, A. K.; Hammer, D. A.; Bates, F. S.; Discher, D. E. Macromolecules 2002, 35, (21), 8203-8208) notably PEO(2k)-b-PCL(12k) possesses a calculated fraction (see, Brandrup, J.; Immergut, E. H.; Grulke, Z. A., Polymer Handbook; John Wiley & Sons: New York, 1999; Vol. 6, p 53) which is significantly lower (−0.15).

PEO(2k)-b-PCL(12k)-based polymersomes and other polymersomes of the invention constitute a unique biodegradable delivery vehicle that combines elements of both reservoir and monolithic diffusion-controlled delivery devices. The large membrane core thickness of PEO(2k)-b-PCL(12k)-based polymersomes (22.5+/−2.3 nm) affords the opportunity to incorporate both hydrophobic (membrane sequestered) and hydrophilic (internal aqueous core) compounds within a single complex vesicular template. Release from these bioresorbable polymersomes will depend upon PCL matrix erosion as well as intrinsic drug permeability from the aqueous core through the membrane. Finally, the self-assembled vesicular architecture allows for facile and economic generation of mesoscopic (nanometer to micron) colloidal devices, enabling large-scale production while eliminating the need for organic co-solvent removal post-assembly.

The surface of the block copolymersome can be modified. In some embodiments, the terminal end of the water-soluble polyethylene oxide is the most attractive location for substitution, due to the specificity of the location for chemical modification and the subsequent availability of the substituted ligand for physical interaction with a surface. Numerous chemical transformations are possible beginning with the terminal alcohol (see, for example, Streitweiser A, Heathcock C H, Kosower E M. Introduction to Organic Chemistry, New York: Macmillan Publishing Co. (1992)). For example, reaction of PEO-OH with acrylonitrile and subsequent protonation converts the terminal group to a primary amine. The reaction conditions are typically compatible with the overall polymer chemistry, and several such reactions will be optimized for application to the block copolymers described below. Techniques for attaching biological molecules to a wide variety of chemical functionalities have been catalogued by Hermanson, et al., Immobilized Affinity Ligand Techniques, New York, N.Y.: Academic Press, Inc. (1992).

Some vesicles have a terminally linked compound that is used as a targeting moiety to specifically bind with a biological situs. In some embodiments, the targeting moiety specifically binds with a biological situs under physiological conditions. Targeting moieties include antibodies, antibody fragments, and substances specific for a given receptor binding site. The receptor binding site, or targeting moiety comprises a receptor-specific peptide, carbohydrate, protein, lipid, nucleoside, peptide nucleic acid, or combinations thereof.

The targeting moiety is optionally attached to the vesicle or polymersome by a linking group. Suitable linking groups are those that provide a desired degree of flexibility without any detrimental effects to the polymersome/imaging agent system.

In some embodiments, the vesicle can additionally comprise a protein, peptide, saccharide, nucleoside, inorganic compound, or organic compound covalently linked to the terminal hydrophilic end of the block copolymer.

In some embodiments, the vesicle additionally comprises a protein, peptide, saccharide, nucleoside, inorganic compound, or organic compound compartmentalized within the aqueous polymersome interior. In other embodiments, the vesicle additionally comprises a protein, peptide, saccharide, nucleoside, inorganic compound, or organic compound compartmentalized within the hydrophobic vesicle membrane.

The compartmentalized agent, in some embodiments, is of therapeutic value within the human body. In some embodiments, the vesicle contains compartmentalized or covalently integrated components approved by the United States Food and Drug Administration (FDA) for use in vivo.

Some vesicles additionally comprising at least one of an emissive agent, a cytotoxic agent, a magnetic resonance imaging (MRI) agent, positron emission tomography (PET) agent, radiological imaging agent or a photodynamic therapy (PDT) agent compartmentalized within the hydrophobic vesicle membrane. In some embodiments, the emissive agent compartmentalized within the hydrophobic vesicle membrane. In other embodiments, the MRI agent compartmentalized is within the hydrophobic vesicle membrane. In certain embodiments, the PET agent compartmentalized within the hydrophobic vesicle membrane. Some compositions have at least one radiological imaging agent compartmentalized within the hydrophobic vesicle membrane. Some compositions have at least one PDT agent compartmentalized within the hydrophobic vesicle membrane. In other embodiments, the agents are compartmentalized the aqueous polymersome interior.

The vesicles can additionally comprising at least one of a secondary emissive agent, a cytotoxic agent, a magnetic resonance imaging (MRI) agent, positron emission tomography (PET) agent, radiological imaging agent or a photodynamic therapy (PDT) agent compartmentalized within the hydrophobic vesicle membrane. In some embodiments, the secondary emissive agent is compartmentalized within the aqueous polymersome interior.

In some embodiments, polymersomes of the invention of contain at least one visible- or near infrared-emissive agent that is dispersed within the polymersome membrane. Some emissive agents emit light in the 700-1100 nm spectral regime.

Certain emissive agents comprise a porphyrin moiety. In some embodiments, the emissive agent comprises at least two porphyrin moieties where the porphyrin moieties are linked by a hydrocarbon bridge comprising at least one unsaturated moiety.

Some emissive agents useful in the invention are porphycene, rubyrin, rosarin, hexaphyrin, sapphyrin, chlorophyl, chlorin, phthalocynine, porphyrazine, bacteriochlorophyl, pheophytin, texaphyrin macrocyclic-based components, or metalated derivatives thereof.

Useful emissive agents include emissive agents that are a laser dye, fluorophore, lumophore, or phosphor.

Suitable laser dyes include p-terphenyl, sulforhodamine B, p-quaterphenyl, Rhodamine 101, curbostyryl 124, cresyl violet perchlorate, popop, DODC iodide, coumarin 120, sulforhodamine 101, coumarin 2, oxozine 4 perchlorate, coumarin 339, PCM, coumarin 1, oxazine 170 perchlorate, coumarin 138, nile blue A perchlorate, coumarin 106, oxatine 1 perchlorate, coumarin 102, pyridine 1, coumarin 314T, styryl 7, coumarin 338, HIDC iodide, coumarin 151, PTPC iodide, coumarin 4, cryptocyanine, coumarin 314, DOTC iodide, coumarin 30, HITC iodide, coumarin 500, HITC perchlorate, coumarin 307, PTTC iodide, coumarin 334, DTTC perchlorate, coumarin 7, IR-144, coumarin 343, HDITC perchlorate, coumarin 337, IR-NO, coumarin 6, IR-132, coumarin 152, IR-125, coumarin 153, boron-dipyrromethere, HPTS, flourescein, rhodamine 110, 2,7-dichlorofluorescein, rhodamine 65, and rhodamin 19 perchlorate, rhodamine b, where said laser dye is modified by addition of a hydrophobic substitutent, said laser dye being substantially within the polymersome membrane.

Some compositions have at least one emissive agent is a near infrared (NIR) emissive species that is a di- and tricarbocyanine dye, croconium dye, thienylenephenylenevinylene species substituted with at least one electron withdrawing substituent, where said emissive species is modified by addition of a hydrophobic substitutent, said laser dye being substantially within the polymersome membrane.

Certain emissive agents are emissive conjugated compounds having at least two covalently bound moieties; whereby upon exposing the compound to an energy source for a time and under conditions effective to cause the compound to emit light at a wavelength between 700-1100 nm, is of an intensity that is greater than a sum of light emitted by either of covalently bound moieties individually.

The emissive agent can be an emissive conjugated compound comprising at least two covalently bound moieties; whereby upon exposing the compound to an energy source for a time and under conditions effective to cause the compound to emit light that at a wavelength between 700-1100 nm, and exhibits an integral emission oscillator strength that is greater than the emission oscillator strength manifest by either one of the said moieties individually.

Some emissive agents have covalently bound moieties that define the emissive species are linked by at least one carbon-carbon double bond, carbon-carbon triple bond, or a combination thereof. The covalently bound moieties that define the emissive species can be linked by ethynyl, ethenyl, allenyl, butadiynyl, polyvinyl, thiophenyl, furanyl, pyrrolyl, or p-diethylylarenyl linkers or by a conjugated heterocycle that bears diethynyl, di(polyynynyl), divinyl, di(polyvinvyl), or di(thiophenyl) substituents. In certain embodiments, the covalently bound moieties that define the emissive species are linked by at least one imine, phenylene, thiophene, or amide, ether, thioether, ester, ketone, sulfone, or carbodiimide group.

Some vesicles contain a phorphinato imaging agent is an ethynyl- or butadiynyl-bridged multi(porphyrin) compound that features a β-to-β, meso-to-β, or meso-to-meso linkage topology, and the porphinato imaging agent being capable of emitting in the 600-to-1100 nm spectral regime. Some suitable porphyrin-based imaging agents are of the formula:

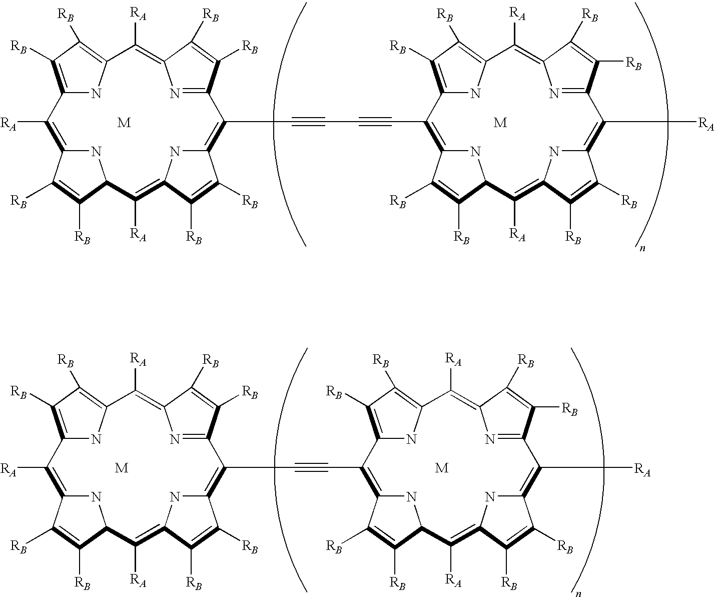

where M is a metal or H₂, where H₂ denotes the free ligand form of the macrocycle; R_{A} and R_{B} are each, independently, H, C₁-C₂₀ alkyl or C₁-C₂₀ heteroalkyl, C₆-C₂₀ aryl or heteroaryl, C(R_{C})═C(R_{D})(R_{E}), C≡C(R_{D}), or a chemical functional group comprising a peptide, nucleoside or saccharide where R_{C}, R_{D} and R_{E} are each independently, H, F, Cl, Br, I, C₁-C₂₀ alkyl or C₄-C₂₀ heteroalkyl, aryl or heteroaryl, C₂-C₂₀ alkenyl or heteroalkenyl, alkynyl or C₂-C₂₀ heteroalkynyl, trialkylsilyl, or porphyrinato; and n is an integer from 1 to 10. In some embodiments, n is an integer from 1 to 8.

M of the porphyrin-based imaging agent can be zinc, magnesium, platinum, palladium, or H₂, where H₂ denotes the free ligand form of the macrocycle.

In some embodiments, the polymersome porphyrin-based imaging agent is emissive. Some agents are multi(porphyrin) imaging agent comprises a meso-to-meso ethyne- or butadiyne-bridged linkage topology, the imaging agent being capable of emitting in the 600-to-1100 nm spectral regime.

Those skilled in the art will recognize the wide variety of dimers, trimers, oligomers or polymers that can be prepared from the porphyrin-containing compounds. Such compounds can be found, for example, in U.S. patent application Ser. No. 10/777,552, the disclosure of which is incorporated herein in its entirety.

The invention is intended to be illustrated but not limited by the following examples.

### EXPERIMENTAL

### Materials

ε-Caprolactone (ε-CL) was purchased from Aldrich, dried over calcium hydride (CaH₂) at room temperature for 48 h, and distilled under reduced pressure. Monomethoxyl poly(ethylene oxide) (MePEO) homopolymers, with one terminal —OH group and Mₙ of 5000, 2000, 1100 and 750, were purchased from Fluka. Larger MePEO blocks (Mₙ=1100, 2000 and 5000) were purified by dissolution in tetrahydrofuran (THF), followed by precipitation into ether, and subsequent drying at 40° C. and 10 mmHg for 24 h. Other MePEO blocks (M_{w}=750) were used as received. Stannous(II) octonate (SnOct₂) was purchased from Sigma and used as received. Ethylene oxide (EO) was purchased from Aldrich, purified by passage through potassium hydroxide, condensed onto CaH₂ while stirring for 2 h, and finally collected by distillation. Naphthalene was recrystallized from ether before use and THF was distilled over Na mirror. Other chemicals were commercially available and used as received.

### Polymerization Reactions

Ring-opening polymerization: Monomethoxyl poly(ethylene oxide) (MePEO) was filled into a flamed flask under argon. Caprolactone monomer (with various calculated weight ratios to MePEO) was then injected into the flask via syringe and two small drops of SnOct₂ were added to the reaction mixture. The flask was connected to a vacuum line, evacuated, sealed, and immersed in an oil bath at 130° C. A progressive increase in viscosity of the bulk homogeneous mixture was always observed during polymerization. Copolymers were collected after 24 h upon cooling of the reaction mixture to room temperature. The resultant block copolymers were dissolved in methylene chloride and precipitated in excess cold methanol/hexane (4° C.). The white powder products were obtained and dried at 40° C. under vacuum for more than two days.

Anionic living polymerization: In a flame-dried and argon-purged flask, 30 mL of anhydrous THF, 0.55 mL (10 mmol) acetonitrile, and 5 mL potassium naphthalene/THF solution (1 mmol/mL) were added under argon stream. After vigorous stirring (70 min at 20° C.), the mixture was cooled in an ice-water bath and distilled EO was added by cool syringe. After 48 h polymerization at room temperature, a sample of reaction product (approx 5 mL CN-PEO) was removed, treated with an acetone solution containing acetic acid, precipitated with excess diethyl ether, and dried under vacuum at room temperature. Subsequently, ε-caprolactone, dissolved in THF at a calculated mole ratio of CL/EO, was added to the remaining reaction mixture of CN-PEO. After 5-10 min at 0° C., the polymerization was quenched by adding excess acetone solution containing acetic acid; the final copolymer was recovered by precipitation in diethyl ether and dried under vacuum at 40° C. for two days.

### Copolymer Characterization

PEO polymers and copolymers were characterized by ¹H-NMR (proton-nuclear magnetic resonance) spectroscopy using Bruker 300 MHz or 500 Mhz NMR instruments. Deuterated chloroform (CDCl₃) was used as the solvent and tetramethylsilane (TMS) as the internal standard. Weight-average molecular weight (M_{w}) values and the polydispersity index (M_{w}/Mₙ) for each copolymer formulation were determined by GPC (RAININ HPXL), at room temperature (25° C.) using two separate columns (PLgel 5μ Mixed, 300×7.5 mm), via dynamic laser scattering and refractive index detectors. THF was utilized as the eluting solvent. PEO standards were used to calibrate the molecular weights of the copolymers from refractive index data.

### Preparation of Polymersomes

Two preparation methods, self-assembly via thin-film hydration and vesicle formation via organic co-solvent/aqueous extraction, were employed to assemble the PEO-b-PCL copolymers into various aqueous morphologies. Film hydration has been extensively utilized for preparing non-degradable polymersomes comprised of PEO-b-PBD and PEO-b-PEE diblock copolymers. See, Discher, D. E.; Eisenberg, A. Science, 2002, 297, 967-973 and Ghoroghchian, P. P.; Frail, P. R.; Susumu, K.; Blessington, D.; Brannan, A. K.; Bates, F. S.; Chance, B.; Hammer, D. A.; Therien, M. J. Proceedings of the National Academy of Sciences of the United States of America (PNAS), 2005, 102, 2922-2927. In brief, 200 microliters of 5-10 mg/mL PEO-b-PCL copolymer solution in chloroform (with and without 1 mol % Nile Red) were uniformly coated on the surface of a roughened Teflon® plate followed by evaporation of the solvent under vacuum for >12 h. Addition of aqueous solution (e.g. 250-300 milliosmolar sucrose or PBS), and heating at 60° C. for 48 h, led to spontaneous budding of giant (5-20 μm) biodegradable polymersomes, off of the Teflon®-deposited thin-film, into the aqueous solution. PBS is an aqueous buffer solution that is 50 mM Na₂BPO₄ and 140 mM NaCl with a pH of 7.2. Nile red was incorporated into the polymersome membranes during self-assembly and enabled facile visualization of resultant copolymer aqueous morphology via confocal fluorescence microscopy.

Small (<300-nm diameter) unilamellar polymersomes that possess appropriately narrow size distributions were prepared via procedures analogous to those used to formulate small lipid vesicles (sonication, freeze-thaw extraction, and extrusion). See, Ghoroghchian, P. P.; Frail, P. R.; Susumu, K.; Blessington, D.; Brannan, A. K.; Bates, F. S.; Chance, B.; Hammer, D. A.; Therien, M. J. Proceedings of the National Academy of Sciences of the United States of America (PNAS), 2005, 102, 2922-2927. The sonication procedure involved placing a sample vial containing the aqueous-based solution and a dried thin-film formulation (of polymer uniformly deposited on a polytetrafluoroethylene (PTFE) film such as a Teflon® film) into a bath sonicator (Fischer Scientific; Model FS20) with constant agitation for 30 min. Several cycles of freeze-thaw extraction followed by placing the sample vials (containing solutions of 300 nm-500 nm diameter polymersomes) in liquid N₂. Once the bubbling from the liquid N₂ subsided, the vials were subsequently transferred to a 60° C. water bath. Extrusion to a mono-dispersed suspension of small (e.g., 100-nm diameter) vesicles proceeded by introduction of the polymersome solution into a thermally controlled stainless steel cylinder connected to pressurized nitrogen gas. The size distributions of the PEO-b-PCL suspensions were determined in each case by dynamic light scattering.

For the co-solvent/water extraction method, the diblock copolymers were dissolved in chloroform or tetrahydrofuran (THF) (at 10 mg/mL) and introduced at 1:100 vol % into aqueous solution (sucrose, PBS or benzene/alcohol aqueous solution) via organic co-solvent injection. The various structures formed from these diblock copolymers were extracted from the solvent mixture by aqueous dialysis (for organic co-solvent removal) at room temperature for 24 hours.

### Incorporation of Hydrophobic and Hydrophilic Compounds Within PEO-b-PCL-Based Vesicles

Aqueous insoluble compounds (e.g. Nile Red dye) are incorporated within the hydrophobic membrane of PEO-b-PCL-based vesicles by first co-dissolving them with the bulk polymer in the same organic solution (typically chloroform, methylene chloride, tetrahydrofuran, ethanol, methanol, or a combination thereof). The organic solution (containing the hydrophobic molecule and polymer are then dried as a thin-film on Teflon®. Upon aqueous hydration of the thin-film, and heating above the melting temperature of the polymer (>52° C.), vesicles are formed in aqueous solution containing the aqueous-insoluble compound within their thick lamellar membranes (see for e.g. Nile Red incorporation within PEO(2k)-b-PCL(12k)-based polymersomes in FIGS. 2 and 3).

Encapsulation of hydrophilic compounds (e.g. calcein dye) within the aqueous milieu of PEO-b-PCL-based polymersomes occurs upon their introduction into the aqueous solution used to hydrate the dry thin-film formulation of the diblock copolymer on Teflon. After vesicle self-assembly, the unincorporated portion of the agent can be removed from the external solution surrounding the vesicles by aqueous dialysis (see FIG. 2 depicting aqueous-soluble calcein dye within the interior milieu of PEO(2k)-b-PCL(12k)-based polymersomes).

### Reduction of Vesicle Size

Small vesicles that posses appropriately narrow size distributions (within the 100-200 μm diameter range) can be prepared via procedures analogous to those used to formulate small unilamellar liposomes (sonication, freeze-thaw extraction, and extrusion).

The sonication procedure involves placing a sample vial containing the aqueous-based solution and a dried thin-film formulation (of polymer and NIRF species uniformly deposited on Teflon) into a bath sonicator (Fischer Scientific, Fair Lawn, N.J.; Model FS20) with constant agitation for 30 minutes. Several (×x 3-5) cycles of freeze-thaw extraction follow by placing the sample vials (containing solutions of medium-sized, 300 nm, NIR-emissive polymersomes) in liquid N₂. Once the bubbling from the liquid N₂ subsides, the vials are subsequently transferred to a 56° C. water bath.

Extrusion to a mono-dispersed suspension of small (100 nm diameter) vesicles proceeds by the introduction of the aqueous solution into a thermally controlled, stainless steel, cylinder connected to pressurized nitrogen gas. The vesicle solution is pushed through a 0.1 μm polycarbonate filter (Osmonics, Livermore, Calif.) supported by a circular steel sieve at the bottom of the cylinder, where the vesicle solution is collected after extrusion. This procedure can be repeated multiple times, and the size distribution of vesicles is measured by dynamic light scattering (DynaPro, Protein Solutions, Charlottesville, Va.).

### Characterization of Thermal Transitions of PEO-b-PCL in Bulk and Within Aqueous Vesicle Suspensions

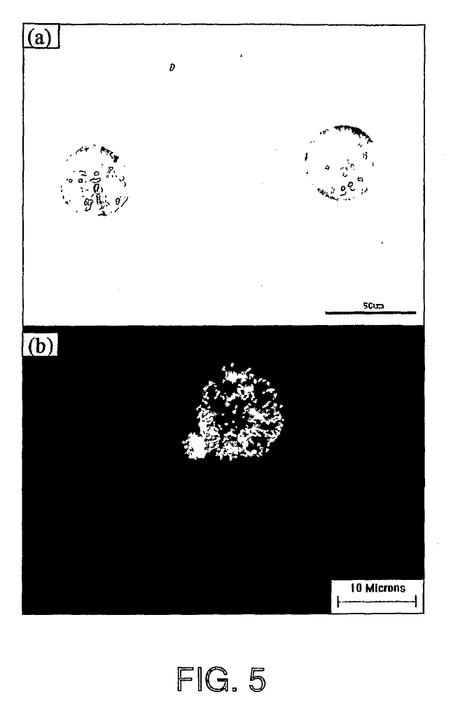

Differential scanning calorimetry (DSC; TA instruments Q100, New Castle Del.) was utilized to elucidate the thermal transitions of PEO(2k)-b-PCL(12k) in bulk and within aqueous vesicle solutions (FIG. 5). FIG. 5A shows two distinct first-order transitions in bulk PEO(2k)-b-PCL(12k) consistent with a diblock copolymer comprised of two crystallizable homopolymers (onset of melting=52.6° C.; total heat of transition=90.2 J/g). See, Bogdanov, B.; Vidts, A.; Van Den Bulcke, A.; Verbeeck, R.; Schacht, E. Polymer 1998, 39, (8-9), 1631-1636 and Gan, Z. H.; Jiang, B. Z.; Zhang, J. J. Appl. Poly. Sci. 1996, 59, (6), 961-967. Immediately upon dissolution of the dry polymer in water (at 30 mg/mL), the double melting peaks were transformed to a single first-order transition with a peak melting temperature at 52.3° C. upon second heating (onset of melting=48.8° C.; heat of transition=33.2 J/g; FIG. 5B). The polymer showed no further changes in its thermal transitions upon aqueous dissolution and isothermal heating at 60° C. for 48 h. DSC of PEO(2k)-b-PCL(12k)-based polymersomes, whose structure was observed by cryogenic transmission electron microscopy (cryo-TEM) and size distributions determined by dynamic light scattering (DLS), displayed the same first-order transition upon heating irrespective of vesicle size (onset of melting=48° C.; peak=52° C.; heat of transition=43 J/g). The isothermal crystallization and melting behavior of bulk PEO-b-PCL has been previously studied by WAXD, SAXS, and DSC, which demonstrated that despite strong crystallizablility in the PEO homopolymer, only the PCL block in the PEO-b-PCL copolymer is crystallizable when the PEO weight fraction is less than 20%. See, Gan, Z. H.; Jiang, B. Z.; Zhang, J. J. Appl. Poly. Sci. 1996, 59, (6), 961-967. As such, it is strongly suggestive that in PEO(2k)-b-PCL(12k)-based vesicles, the membrane consists entirely of a PCL lamella with a PEO corona facing the external solution and internal aqueous milieu.

### Characterization of PEO-b-PCL Morphology in Dilute Aqueous Solution

Confocal laser scanning microscopy (BioRad Radiance 2000) and epifluorescent optical microscopy (Zeiss Axiovert 200) were employed to characterize the self-assembled aqueous morphology of PEO-b-PCL incorporating fluorescent Nile Red (1 mol % dye to polymer). The microscopes were equipped with appropriate excitation and emission filters for the dye.

### Synthesis and Characterization of Biodegradable PEO-b-PCL Diblock Copolymers

A series of PEO-b-PCL diblock copolymers were synthesized via ring-opening polymerization of ε-caprolactone and commercially available MePEO (Mₙ=5000, 2000, 1100 and 750). MePEO with one hydroxyl end group was used as the macroinitiator to activate the polymerization (at 130° C. for 24 h) of ε-CL monomer under catalysis of stannous(II) octoate (SnOct₂) (Scheme 1). PEO-b-PCL diblock copolymers have been previously synthesized by utilizing a number of different catalyst systems (see Meng F.; Hiemstra, C.; Engbers, G. H. M.; Feijen J. Macromolecules, 2003, 36, 3004-3006, Zastre, J.; Jackson, J.; Bajwa, M.; Liggins, R.; Iqbal, F.; Burt, H. European Journal of Pharmaceutics and Biopharmaceutics, 2002, 54, 299-309, 20, Bogdanov, B.; Vidts, A.; Van Den Bucke, A.; Verbeeck, R.; Schacht, E. Polymer, 1998, 39, 1631-1636, and Hsu, S.; Tang, C.; Lin, C. Biomaterials, 2004, 25, 5593-5601.) as well as with catalysts-free schemes (see, Jeong, Y.; Kang, M.; Sun, H.; Kang, S.; Kim, H.; Moon, K.; Lee, K.; Kim, S.; Jung, S. International Journal of Pharmaceutics, 2004, 273, 95-107 and Cerri, P.; Tricoli, M.; Andruzzi, F.; Paci, M. Polymer, 1989, 30, 338-343). SnOct₂ is the most widely used catalyst for the production of biodegradable polyesters because it is commercially available, easy to handle, soluble in common organic solvents and cyclic ester monomers, and is a permitted food additive in numerous countries (see, Dong, C.; Qiu, K.; Gu, Z.; Feng, X. Macromolecules, 2001, 34, 4691-4696). Furthermore, non-catalyzed ring-opening polymerization of CL must be carried out at high temperature (≧180° C.) for a several days. As such, SnOct₂ was utilized in our system as the catalyst for the synthesis of PEO-b-PCL copolymer under mild reaction conditions. All PEO-b-PCL diblock copolymers synthesized by this ring-opening polymerization are listed in Tables 1-3.

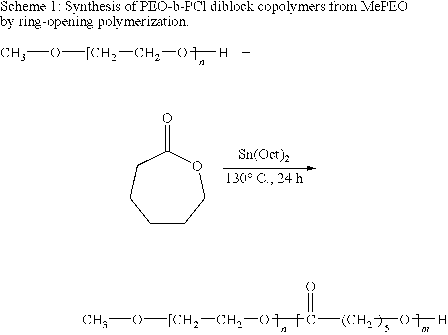

Although the synthesis of PEO-b-PCL copolymer from MePEO via ring-opening polymerization of ε-caprolactone is rather facile, it is difficult to obtain copolymer with highly controllable PEO block molecular weights due to the limited commercial availability of MePEO homopolymers. As an alternative strategy, we utilized anionic living polymerization: the procedure starts from ethylene oxide monomer followed by caprolactone polymerization and offers another route for synthesis of PEO-b-PCL copolymer, yielding a diverse range of available PEO block molecular weights. Moreover, the copolymers' terminal end group (on the PEO block) can be tailored and easily varied by this synthesis strategy. The employed strategy for the synthesis of PEO-b-PCL diblock copolymer, containing an N-terminal group, via anionic living polymerization is depicted in Scheme 2.

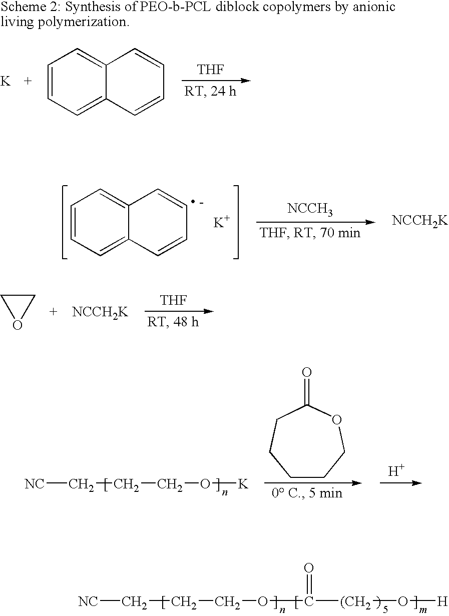

Potassium naphthalenide was synthesized following previously established methodology (see Hillmyer, M. A.; Bates, F. S. Macromolecules, 1996, 29, 6994-7002 and Cammas, S.; Nagasaki, Y.; Kataoka, K. Bioconjugate Chem., 1995, 6, 226-230). Cynomethyl potassium was then was prepared, by metalation of acetonitrile with potassium naphthalenide in THF (see Nagasaki, Y.; Iijima, M.; Kato, M.; Kataoka, K. Bioconjugate Chem., 1995, 6, 702-704 and Deng, M.; Wang, R.; Rong, G.; Sun, J.; Zhang, X.; Chen X.; Jing, X. Biomaterials, 2004, 25, 3553-3558), and utilized as the macro-initiator for ethylene oxide polymerization. Previously, only low molecular weight (high PEO weight fraction) PEO(2.2k)-b-PCL(1.2k) has been synthesized by this anionic living polymerization strategy (see Deng, M.; Wang, R.; Rong, G.; Sun, J.; Zhang, X.; Chen X.; Jing, X. Biomaterials, 2004, 25, 3553-3558). We successfully used the revised reaction conditions (see Scheme 2) to synthesize PEO-b-PCL with a series of PEO block molecular weights (i.e. 1.5 k, 2.2 k, 2.6 k, 3 k, 3.8 k, and 5.8 k), low PEO weight fractions (9.9˜23.4%), and a wide range of copolymer Mₙ (7.8 k-47 k).

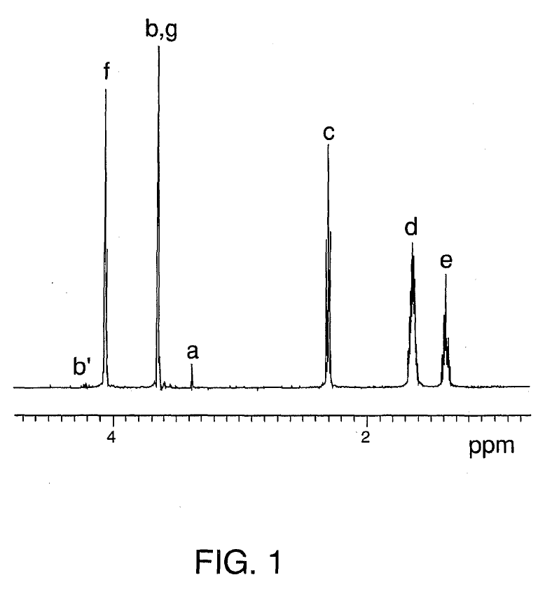

¹H-NMR spectroscopy has been a proven and very useful technique for the characterization of chemical structure and number-average molecular weight of PEO homopolymer and PEO-b-PCL diblock copolymers with different terminal end groups (see Meng F.; Hiemstra, C.; Engbers, G. H. M.; Feijen J. Macromolecules, 2003, 36, 3004-3006, Zastre, J.; Jackson, J.; Bajwa, M.; Liggins, R.; Iqbal, F.; Burt, H. European Journal of Pharmaceutics and Biopharmaceutics, 2002, 54, 299-309, Jeong, Y.; Kang, M.; Sun, H.; Kang, S.; Kim, H.; Moon, K.; Lee, K.; Kim, S.; Jung, S. International Journal of Pharmaceutics, 2004, 273, 95-107, Cerri, P.; Tricoli, M.; Andruzzi, F.; Paci, M. Polymer, 1989, 30, 338-343, Bogdanov, B.; Vidts, A.; Van Den Bucke, A.; Verbeeck, R.; Schacht, E. Polymer, 1998, 39, 1631-1636, Hsu, S.; Tang, C.; Lin, C. Biomaterials, 2004, 25, 5593-5601, and Nagasaki, Y.; Iijima, M.; Kato, M.; Kataoka, K. Bioconjugate Chem., 1995, 6, 702-704). A typical NMR spectrum for MePEO-b-PCL is shown in FIG. 1. The appearance of a small peak around 4.20 ppm (b′), consistent with the terminal methylene end group of the PEO block, in the ¹H-NMR spectrum indicates that the final reaction products were limited to only diblock copolymers of PEO and PCL. A small sharp peak at 3.38 ppm and a very strong peak at 3.65 ppm were attributed to methyl (a, CH₃O-terminated PEO) and methylene groups (b, repeat unit of MePEO), respectively. Peaks at 2.23 ppm, 1.63 ppm, 1.38 ppm and 4.06 ppm were assigned to protons in PCL repeat units (c, d, e, and f methylene). Peaks at 3.65 ppm, from methylene protons of the PEO block, and a triplet at 2.23 ppm, from the methylene protons of the caprolactone repeating units in PCL block (b, COCH₂CH₂CH₂CH₂CH₂O), were used to establish the degree of PCL block polymerization and Mₙ(NMR). ¹H-NMR spectroscopy was further utilized to characterize the number-average molecular weight of PEO, from the calculated ethylene oxide repeat unit number, by comparison to the proton peaks of the end groups (i.e. CH₃O— or CNCH₂CH₂—). The only differences in ¹H-NMR spectra between CN-PEO-b-PCL and MePEO-b-PCL diblock copolymers consists of two weak peaks around 2.50 ppm (the first methylene attached to CN, CN—CH₂—CH₂) and 1.90 ppm (the second methylene for CN—CH₂—CH₂) for the PEO end groups of CN-PEO-b-PCL, replacing the weak peaks at 3.38 ppm (CH₃O—) (PEO block end group seen in MePEO-b-PCL). Number-average molecular weight values of CN-PEO-b-PCL diblock copolymers were also calculated from the NMR spectra.

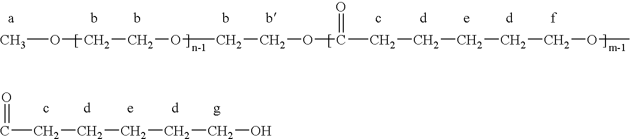

GPC was employed to characterize the molecular weight (M_{w}) and molecular weight distribution (M_{w}/Mₙ) (PDI) of each PEO-b-PCL diblock copolymer formulation. Two types of weight-average molecular weights were calculated from refractive index data by using PEO standard samples and by utilizing dynamic light scattering data, respectively (see Table 1). Some copolymers, such as PEO(5.8k)-b-PCL(24.0k), PEO(5k)-b-PCL(22k), PEO(2k)-b-PCL(12k), and PEO(2k)-b-PCL(15k), exhibited similar molecular weight values as obtained from GPC when compared to those determined from ¹H-NMR. Notably, PEO(5.8k)-b-PCL(33.6k), the largest copolymer synthesized, and PEO(2k)-b-PCL(9.5k), the smallest, however, showed greater differences in the M_{w} determinations from ¹H-NMR vs. GPC data. As PEO-b-PCL diblock copolymer standard samples are not commercially available for the calibration of RI data (GPC), the dn/dc values of the copolymers were obtained from PS standard samples and used to calibrate the DLS data, and thus likely explains the differences in molecular weight value determinations obtained by the two methods (GPC and ¹H-NMR).

From GPC data, PEO-b-PCL diblock copolymers, with various PEO molecular weights (2.2 k, 2.6 k, 3 k, 3.8 k and 5.8 k), synthesized by anionic living polymerization exhibited the narrowest molecular weight distributions overall (PDI: 1.2-˜1.27). PEO-PCL diblock copolymers synthesized from PEO(2k) via ring-opening polymerization showed narrow molecular weight distributions (1.1-1.2) while copolymers derived from PEO(5k) displayed ones that were slightly wider (PDI: 1.32-1.37). Anionic living polymerization therefore provides the best route for the synthesis of PEO-b-PCL diblock copolymers with controlled PEO block molecular weights, various PEO/PCL block ratios, and narrow molecular weight distributions.

TABLE 1
PEO-b-PCL Diblock Copolymer Characterization by GPC vs. ¹H-NMR

| | f_{PEO} | | M_{w}* | PDI | M_{w}* |
|---|---|---|---|---|---|
| PEO-b-PCL copolymers | (Weight) | Mₙ | (GPC, | (GPC, | (GPC, |
| (Block Mₙ from NMR) | (NMR) | (NMR) | DLS) | DLS) | RI) |
| PEO(5.8k)-b-PCL(24.0k) | 19.5% | 29800 | 27800 | 1.24 | 28100 |
| PEO(5.8k)-b-PCL(33.6k) | 14.7% | 39400 | 28500 | 1.20 | 31100 |
| PEO(5k)-b-PCL(22k) | 18.5% | 27000 | 25300 | 1.37 | 24000 |
| PEO(5k)-b-PCL(26k) | 16.1% | 31000 | 31400 | 1.32 | 36200 |
| PEO(3.8k)-b-PCL(17k) | 18.3% | 20800 | 15100 | 1.20 | 18900 |
| PEO(3.8k)-b-PCL(20k) | 16.0% | 23800 | 17600 | 1.25 | 19100 |
| PEO(3.8k)-b-PCL(22.2k) | 14.6% | 26000 | 19200 | 1.26 | 22100 |
| PEO(3k)-b-PCL(16.5k) | 15.4% | 19000 | 16700 | 1.23 | 17300 |
| PEO(3k)-b-PCL(19k) | 13.6% | 22000 | 19400 | 1.24 | 19400 |
| PEO(2.6k)-b-PCL(11.2k) | 18.8% | 13800 | 16200 | 1.27 | 19000 |
| PEO(2.6k)-b-PCL(12.3k) | 17.4% | 14900 | 17300 | 1.25 | 20600 |
| PEO(2.6k)-b-PCL(15.5k) | 14.4% | 18100 | 19600 | 1.25 | 24100 |
| PEO(2k)-b-PCL(9.5k) | 17.4% | 11500 | 12500 | 1.14 | 16300 |
| PEO(2k)-b-PCL(12k) | 14.3% | 14000 | 13700 | 1.21 | 15400 |
| PEO(2k)-b-PCL(15k) | 11.8% | 17000 | 16100 | 1.21 | 18300 |

*M_{w} (GPC, DLS) and PDI were calculated from dynamic light scattering (DLS) data. M_{w} (GPC, RI) values were calculated from refractive index (RI) data and calibrated by PEO standard samples.

### Aqueous Assembly of PEO-b-PCL Diblock Copolymers

Two preparation methods, film hydration and organic co-solvent injection/extraction, were chosen to assemble amphiphilic PEO-b-PCL diblock copolymers into various aqueous morphologies (including polymersomes). As film hydration promotes aqueous self-assembly of the copolymers, and enables facile large-scale generation while obviating the need for post-assembly processing, it was preferentially utilized. Tables 2 and 3 summarize the observed aqueous morphologies of the various PEO-b-PCL diblock copolymer formulations (as determined by incorporation of 1 mol % Nile Red and visualized by fluorescence confocal and optical microscopy).

TABLE 2
Aqueous Morphology of PEO-b-PCL Diblock Copolymers (PEO: 2k-5.8k)

| | f_{PEo} | |
|---|---|---|
| PEO-b-PCL copolymers | (Weight) | |
| (Block Mₙ from NMR) | (NMR) | Preparation Method: Film hydration |
| PEO(5.8k)-b-PCL(22k) | 21.0% | Irregular particles |
| PEO(5.8k)-b-PCL(23.8k) | 19.6% | Few microspheres and many irregular particles |
| PEO(5.8k)-b-PCL(24.0k) | 19.5% | Few microspheres and many irregular particles |
| PEO(5.8k)-b-PCL(30.2k) | 16.1% | All irregular particles |
| PEO(5.8k)-b-PCL(33.6k) | 14.7% | All irregular particles |
| PEO(5.8k)-b-PCL(37.7k) | 13.3% | Some irregular particles |
| PEO(5.8k)-b-PCL(41.2k) | 12.3% | Scant irregular particles |
| PEO(5k)-b-PCL(10k) | 33.3% | Many irregular particles |
| PEO(5k)-b-PCL(16k) | 23.8% | Irregular particles |
| PEO(5k)-b-PCL(22k) | 18.5% | Irregular particles |
| PEO(5k)-b-PCL(26k) | 16.1% | Irregular small and big particles |
| PEO(5k)-b-PCL(32k) | 13.5% | Some irregular particles |
| PEO(5k)-b-PCL(52k) | 8.8% | Scant irregular particles |
| PEO(3.8k)-b-PCL(17k) | 18.3% | Some polymersomes and irregular particles |
| PEO(3.8k)-b-PCL(17.7k) | 17.7% | Few polymersomes and irregular particles |
| PEO(3.8k)-b-PCL(20k) | 16.0% | Few polymersomes and irregular particles |
| PEO(3.8k)-b-PCL(22.2k) | 14.6% | Scant polymersomes and irregular particles |
| PEO(3k)-b-PCL(16.5k) | 15.4% | Few polymersomes and irregular particles |
| PEO(3k)-b-PCL(19k) | 13.6% | Scant polymersomes and irregular particles |
| PEO(3k)-b-PCL(20.5k) | 12.8% | Some irregular particles |
| PEO(3k)-b-PCL(24.7k) | 10.8% | Few irregular particles |
| PEO(3k)-b-PCL(25.8k) | 10.4% | Few irregular particles |
| PEO(2.6k)-b-PCL(11.2k) | 18.8% | Some polymersomes and irregular particles |
| PEO(2.6k)-b-PCL(12.3k) | 17.4% | Few polymersomes and irregular particles |
| PEO(2.6k)-b-PCL(15.5k) | 14.4% | Few polymersomes and irregular particles |
| PEO(2.2k)-b-PCL(7.4k) | 23.4% | Only irregular particles |
| PEO(2k)-b-PCL(7.4k) | 27.0% | Only irregular particles |
| PEO(2k)-b-PCL(9.5k) | 17.4% | Many polymersomes and some irregular particles |
| PEO(2k)-b-PCL(12k) | 14.3% | All polymersomes |
| PEO(2k)-b-PCL(15k) | 11.8% | Scant polymersomes and few irregular particles |
| PEO(2k)-b-PCL(22k) | 8.3% | Scant particles |

Numbers of unique aqueous microscale structures: all (100%) > many (60-80%) > some (30-50%) > few (10-20%) > scant (<5%)

TABLE 3
Aqueous Morphology of PEO-b-PCL Diblock Copolymers (PEO: 750-1.5k)

| | f_{PEO} | |
|---|---|---|
| PEO-b-PCL copolymers | (Weight) | |
| (Block Mₙ from NMR) | (NMR) | Preparation Method: Film hydration |
| PEO(1.5k)-b-PCL(6.3k) | 19.2% | Scant microspheres and mostly irregular particles |
| PEO(1.5k)-b-PCL(10.4k) | 12.6% | Many irregular particles |
| PEO(1.5k)-b-PCL(12.4k) | 10.8% | Some irregular particles |
| PEO(1.5k)-b-PCL(13.7k) | 9.9% | Some irregular particles |
| PEO(1.1k)-b-PCL(2.9k) | 27.1% | Many irregular particles |
| PEO(1.1k)-b-PCL(3.7k) | 22.6% | Some microspheres and many irregular particles |
| PEO(1.1k)-b-PCL(6.3k) | 14.8% | Some microspheres and many irregular particles |
| PEO(1.1k)-b-PCL(7.0k) | 13.5% | Some microspheres and many irregular particles |
| PEO(1.1k)-b-PCL(7.7k) | 12.5% | Irregular particles |
| PEO(1.1k)-b-PCL(9.5k) | 10.4% | Irregular particles |
| PEO(1.1k)-b-PCL(13.0k) | 7.8% | Irregular particles |
| PEO(750)-b-PCL(2850) | 20.8% | Many irregular big and small particles |
| PEO(750)-b-PCL(5790) | 11.4% | Some small irregular particles |
| PEO(750)-b-PCL(9k) | 7.7% | Irregular particles |

Polymersomes were obtained in nearly quantitative yield uniquely from aqueous hydration and self-assembly of PEO(2k)-b-PCL(12k) diblock copolymer (f_{PEO}=14.3%) (confocal fluorescence micrographs, FIG. 2). Some polymersomes were found to coexist with irregular particles in aqueous preparations of PEO(2k-3.8k)-b-PCL(9.5-22.2k) diblock copolymers with wt-f_{PEO} ranging between 11.8 and 18.8%. Unlike in conventional vesicle-generating PEO-b-PBD copolymers, the range of f_{PEO} (11.8-18.8%), PEO block size (2 k-3.8 k), and total diblock Mₙ (1.5 k to 26 k) in polymersome-forming PEO-b-PCL diblock copolymers is very narrow. In aqueous suspensions of PEO-b-PCL diblock copolymers derived from higher (5 k or 5.8 k) or lower (750-1.5 k) molecular weight PEO blocks, no polymersomes were observed irrespective of PEO/PCL ratio.

PEO-b-PCL polymersomes generated from film hydration either possessed unilamellar (FIG. 2d) or multilamellar (FIG. 2c) membranous structures. Although PEO(2k)-b-PCL(9.5k) had a very narrow molecular weight distribution (PDI:1.1) when compared to PEO(2k)-b-PCL(12k) (PDI:1.2), the yield of polymersomes obtained from this diblock copolymer formulation was significantly lower. Moreover, the polydispersity index of the copolymers seemed to have little influence on polymersome formation. Small polymersomes (100 nm in diameter) could be made by aqueous sonication of a dry thin-film formulation of PEO-b-PCL on Teflon followed by several (×3) cycles of freeze/thaw extraction and membrane extrusion. These small unilamellar polymersomes were characterized by cryo-TEM and the membrane thickness of those derived from PEO(2k)-b-PCL(12k) diblock copolymer was found to be 22.5+/−2.3 nm.

In order to elucidate the effects of diblock copolymer molecular weight distribution on vesicle formation, PEO-b-PCL diblock copolymers with varying PEO block size (2.6 k, 3 k or 3.8 k) and very narrow molecular distributions (PDI=1.1) were separated by GPC and used to generate polymersomes. No further improvement in the yield of vesicles from these samples, however, was observed. Additionally, while PEO(5.8k)-b-PCL(24k) has been previously shown to form vesicles via a solvent injection technique, no polymersomes were observed in aqueous suspensions of this diblock (PDI=1.2) formed via thin-film hydration. Furthermore, many polymersomes (FIG. 4) were obtained from mixtures of PEO-b-PCL diblock copolymers with much wider molecular weight distributions. As such, it was determined that molecular weight distribution had almost no influence on biodegradable polymersomes formation from PEO-b-PCL diblock copolymers.

As PEO-b-PCL diblock copolymer with low PEO weight fraction (<12%) was found to be strongly adherent to the Teflon® film (following aqueous hydration), an organic co-solvent water injection/extraction method was employed in an attempt to prepare polymersomes from a small subset of these copolymers. While no polymersomes were obtained by this extraction method, some perfect microspheres (filled spherical particles) were seen upon organic co-solvent removal via dialysis; the typical morphology of these particles is depicted in the fluorescent micrograph (FIG. 4). The spherical particles seemed to possess porous surfaces (FIG. 4a) and effectively encapsulated Nile Red (FIG. 4b).

TABLE 4
Morphology of PEO-b-PCL diblock copolymers in aqueous suspensions obtained via
film hydration (self-assembly) or organic co-solvent injection/extraction

| | f_{PEO} | | |
|---|---|---|---|
| PEO-b-PCL copolymers | (Weight) | Method: | Method: Organic Co- |
| (Block Mₙ from NMR) | (NMR) | Film Hydration | Solvent Extraction |
| PEO(5k)-b-PCL(10k) | 33.3% | Irregular particles | Small particles |
| PEO(5k)-b-PCL(16k) | 23.8% | Irregular particles | Small particles |
| PEO(5k)-b-PCL(22k) | 18.5% | Irregular particles | microspheres |
| PEO(5k)-b-PCL(52k) | 8.8% | Scant particles | microspheres |
| PEO(2k)-b-PCL(15k) | 11.8% | Scant polymersomes | t microspheres |
| PEO(2k)-b-PCL(22k) | 8.3% | Scant particles | microspheres |

These examples present a series of PEO-b-PCL diblock copolymers varying in PEO block size (Mₙ: 750, 1100, 2000 and 5000), wt-f_{PEO} (7.7%-33.3%), and Mₙ (3.6 k-57 k) were synthesized by ring-opening polymerization of ε-caprolactone monomer using commercially available MePEO as the macro-initiator. Anionic living polymerization was also employed to synthesize PEO-b-PCL copolymers with a wider range of controlled PEO block sizes (Mₙ: 1500, 2200, 2600, 3000, 3800, and 5800), CN as the PEO block terminal group, wt-f_{PEO}=9.9-23.4%, and Mₙ ranging between 7.8 k to 47 k. All copolymers were isolated by GPC and possessed appropriately narrow molecular weight distributions (PDI from 1.14 to 1.37). The PEO-b-PCL diblock copolymers were subsequently screened for the ability to assemble into various aqueous morphologies via two separate preparation methods: film hydration and organic co-solvent/water injection/extraction. Polymersomes were obtained in nearly quantitative yield uniquely from PEO(2k)-b-PCL(12k) diblock copolymer (PDI:1.21), via self-assembly, upon hydration of a dry thin-film deposited on Teflon. While only PEO-b-PCL diblock copolymers possessing a PEO block size of 2 k-3.8 k, and wt-f_{PEO} ranging from 11.8-18.8%, were found to assemble into biodegradable polymersomes, the molecular weight distributions of these copolymers had no influence on vesicle generation. Finally, when compared to established methods for vesicle formation by organic co-solvent/water injection/extraction, self-assembly via film hydration proved to be a more facile and efficient method for the preparation of PEO-b-PCL-based biodegradable polymersomes. As such, it should enable a cost-effective and large-scale production of these meso-structured biomaterials for applications such as cosmetic, imaging, and drug delivery applications.

### Incorporation of a Therapeutic Compound in PEO-b-PCL Diblock Copolymers

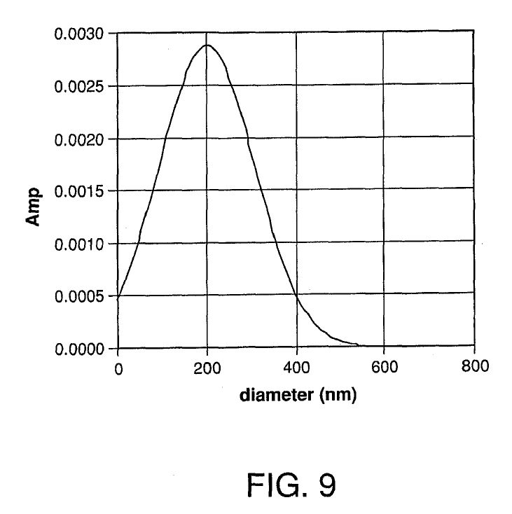

This example shows the loading and release of a therapeutic compound, doxorubicin (DOX), in PEO-b-PCL-based polymersomes. Self-assembly via thin-film hydration was employed in order to form PEO(2k)-b-PCL(12k)-based vesicles. Film hydration has been extensively utilized for preparing non-degradable polymersomes comprised of PEO-b-PBD and PEO-b-PEE diblock copolymers. See Discher, B. M.; Won, Y. Y.; Ege, D. S.; Lee, J. C. M.; Bates, F. S.; Discher, D. E.; Hammer, D. A. Science 1999, 284, (5417), 1143-1146 and Ghoroghchian, P. P.; Frail, P. R.; Susumu, K.; Blessington, D.; Brannan, A. K.; Bates, F. S.; Chance, B.; Hammer, D. A.; Therien, M. J. Proc. Natl. Acad. Sci. USA 2005, 102, (8), 2922-2927. Briefly, 200 microliters of 7 mg/mL PEO(2k)-b-PCL(12k) copolymer solution in methylene chloride were uniformly deposited on the surface of a roughened Teflon plate followed by evaporation of the solvent for >12 h. Addition of aqueous solution (290 milliosmolar ammonium sulfate, pH 5.5) and sonication led to spontaneous budding of biodegradable polymersomes, off the Teflon-deposited thin-film, into the aqueous solution. The sonication procedure involved placing the sample vial containing the aqueous based solution and dried thin-film formulation (of polymer uniformly deposited on Teflon) into a sonicator bath (Branson; Model 3510) with constant agitation for 60 minutes at 65° C. Five cycles of freeze-thaw extraction followed by placing the sample vials in liquid N₂ and subsequently thawing them in a 65° C. water bath. Extrusion using a pressure driven Lipex Thermobarrel Extruder (1.5 mL capacity) at 65° C. was performed to yield small (˜200 nm diameter) unilamellar polymersomes that possess appropriately narrow size distributions. The size distributions of vesicles were determined by dynamic light scattering (see FIG. 9).

Extruded samples were dialyzed in iso-osmotic sodium acetate solution (50 mM sodium acetate, 100 mM sodium chloride, pH˜7.4). Dialysis solutions were changed 3 times over approximately 30 hours. Post-dialysis, doxorubicin was loaded into the polymersomes via an ammonium sulfate gradient. See Haran, G.; Cohen, R.; Bar, L. K.; Barenholz, Y. Biochim. Biophys. Acta 1993, 1151, (2), 201-215; Bolotin, E. M.; Cohen, R.; Bar, L. K.; N., E.; Ninio, S.; Lasic, D. D.; Barenholz, Y. J. Liposome Res. 1994, 4, 455-479; and de Menezes, D. E. L.; Pilarski, L. M.; Allen, T. M. Cancer Res. 1998, 58, (15), 3320-3330. The polymersomes were incubated with doxorubicin in a ratio of 1:0.2 polymer:drug (w/w) for 7 hours at a temperature above their main gel to liquid-crystalline phase transition temperature (65° C.). Aggregation of DOX within the polymersome core led to quenching of its fluorescence emission. Non-entrapped DOX was removed using an Acta Basic 10 HPLC with Frac 950; the solution was passed through a C-1640 column with Sephacryl S500-HR media. The collected DOX-loaded polymersome suspension was centrifuged and concentrated into an approximately 1 mL volume. The vesicles were then aliquoted into various (290 mOsM) solutions buffered at pH˜5.5 (50 mM sodium acetate and 100 mM sodium chloride) and pH˜7.4 (PBS), with N=4 samples for each buffer. Release studies of DOX from the loaded polymersomes were initiated immediately following aliquoting; DOX fluorescence was measured fluorometrically (using a SPEX Fluorolog-3 fluorimeter; λₑₓ=480 nm, λₑₘ=590 nm) at various intervals up to fourteen days. As DOX was released from the polymersome core, and diluted into the surrounding solution, its fluorescence emission increased over time. At the culmination of the study, the samples were solubilized using Triton X-100. The percent release over time was calculated by comparing the measured fluorescence at each time point to final DOX fluorescence, as determined upon solubilization of remaining intact polymersomes with TritonX-100, at the completion of the study.

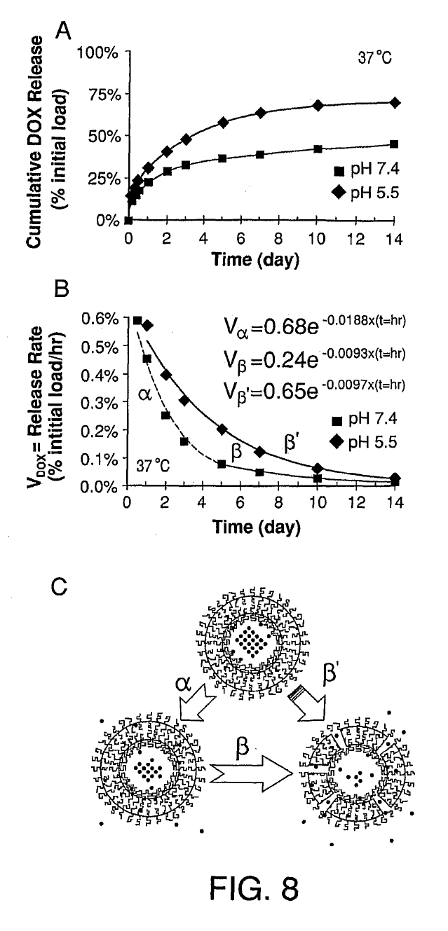

FIG. 8A shows the in situ release of DOX in various physiological conditions (pH 5.5 and 7.4; T=37° C.) which was subsequently monitored fluorometrically (λex=480 nm, λem=590 nm) over 14 days. The release of DOX was measured at two pHs, 5.5 and 7.4, and at 37° C.

Under both conditions, we observed an immediate burst release phase (˜20% of initial vesicle load from 0-8 h), followed by controlled release. The dynamics of release were different at the two pHs. At pH 7.4, release was observed in two distinct phases, denoted α and β, which were well fit by exponential regression analysis (R²=0.99). The α phase (days 1-5, dotted line FIG. 8B) corresponds to a regime where DOX release is predominantly dependent upon the rate of drug permeation through the PCL membrane, dominating the slower rate of matrix erosion (see schematic FIG. 8C). The β phase (days 5-14, solid line FIG. 8B) is consistent with DOX release facilitated predominantly by significant hydrolytic membrane degradation. At pH 5.4, a single phase (β′), is observed. The rate constant for release in this phase is similar to that observed during the beta phase of pH=7.4, indicating that the mechanism of release is similar. DOX release at pH=5.4, however, is more rapid since acid catalyzed hydrolysis of the PCL membrane is the dominant mechanism at short times.

As such, in vivo drug release from these bioresorbable polymersomes will likely depend upon both PCL matrix erosion as well as a drug's intrinsic permeability from the aqueous core through the membrane. Notably, when compared to degradable polymersomes formed from blending “bio-inert” and hydrolysable components (where τ_{1/2}release≈τ_{1/2}circulation˜tens of h) (see, Ahmed, F.; Hategan, A.; Discher, D. E.; Discher, B. M. Langmuir 2003, 19, (16), 6505-6511; Ahmed, F.; Discher, D. E. J. Controlled Release 2004, 96, (1), 37-53; and Photos, P. J.; Bacakova, L.; Discher, B.; Bates, F. S.; Discher, D. E. J. Controlled Release 2003, 90, (3), 323-334.), PEO(2 k)-b-PCL(12 k)-based vesicles possess much slower release kinetics (τ_{1/2}release˜days), offering potential advantages for future intravascular drug delivery applications. Moreover, their large membrane core thickness (22.5+/−2.3 nm) affords the opportunity for facile incorporation of both hydrophobic (membrane sequestered) and hydrophilic (internal aqueous core) compounds within a single complex delivery vehicle. Finally, the self-assembled vesicular architecture allows for facile and economic generation of mesoscopic (nanometer to micron) colloidal devices, enabling large-scale production while eliminating the need for costly removal of organic co-solvents post-assembly.

All patents and publications referenced herein are incorporated in their entirety.

What is claimed:

1. A vesicle comprising a block copolymer of polyethylene oxide and polycaprolactone, the polyethylene oxide having a number average molecular weight from about 2.0 to about 3.8 kD, the block copolymer having a fraction of polyethylene oxide from about 11 to about 20 percent by weight.

2. A vesicle consisting essentially of a block copolymer of polyethylene oxide and polycaprolactone, the polyethylene oxide having a number average molecular weight from about 2.0 to about 3.8 kD, the block copolymer having a fraction of polyethylene oxide from about 11 to about 20 percent by weight.

3. A vesicle comprising a block copolymer in which at least one block is polyethyleneoxide and one block is polycaprolcatone, the polyethylene oxide having a number average molecular weight from about 2.0 to about 3.8 kD, the block copolymer having a fraction of polyethylene oxide of from about 11 to about 20 percent by weight.

4. A vesicle consisting essentially of a block copolymer in which at least one block is polyethyleneoxide and one block is polycaprolcatone, the polyethylene oxide having a number average molecular weight from about 2.0 to about 3.8 kD, the block copolymer having a fraction of polyethylene oxide of from about 11 to about 20 percent by weight.

5. The vesicle of claim 1 wherein fraction of polyethylene oxide of from about 12 to about 19 percent by weight.

6. The vesicle of claim 1 wherein fraction of polyethylene oxide of from about the 11.8 to 18.8 percent by weight.

7. The vesicle of claim 1 wherein the number average molecular weight of the polycaprolactone is from about 9 to about 23 kD.

8. The vesicle of claim 7 wherein the number average molecular weight of the polycaprolactone is from about 9.5 to about 22.2 kD.

9. The vesicle of claim 7 where the molecular weight of the polyethylene oxide is about 2 kD and the molecular weight of the polycaprolactone is about 12 kD.

10. The vesicle of any one of claims 1-4 additionally comprising a protein, peptide, saccharide, nucleoside, inorganic compound, or organic compound compartmentalized within the aqueous polymersome interior.

11. The vesicle of claim 1 additionally comprising a protein, peptide, saccharide, nucleoside, inorganic compound, or organic compound compartmentalized within the hydrophobic vesicle membrane.

12. The vesicle of claim 1 additionally comprising a protein, peptide, saccharide, nucleoside, inorganic compound, or organic compound covalently linked to the terminal hydrophilic end of the block copolymer

13. The vesicle of claim 10 where the compartmentalized agent is of therapeutic value within the human body.

14. The vesicle of claim 11 where the compartmentalized agent is of therapeutic value within the human body.

15. The vesicle of claim 12 where the compartmentalized agent is of therapeutic value within the human body.

16. The vesicle of claim 12 wherein the terminally linked compound is used as a targeting moiety to specifically bind with a biological situs.

17. The vesicle of claim 16 wherein the targeting moiety specifically binds with a biological situs under physiological conditions.

18. The vesicle of claim 16 wherein the targeting moiety comprises an antibody, antibody fragment, or substance specific for a given receptor binding site.

19. The vesicle of claim 16 wherein the receptor binding site or targeting moiety comprises a receptor-specific peptide, carbohydrate, protein, lipid, nucleoside, peptide nucleic acid, or combinations thereof.

20. The vesicle of claim 1 additionally comprising at least one of an emissive agent, a cytotoxic agent, a magnetic resonance imaging (MRI) agent, positron emission tomography (PET) agent, radiological imaging agent or a photodynamic therapy (PDT) agent compartmentalized within the hydrophobic vesicle membrane.

21. The vesicle of claim 1 additionally comprising at least one of an emissive agent compartmentalized within the hydrophobic vesicle membrane.

22. The vesicle of claim 1 additionally comprising at least one MRI agent compartmentalized within the hydrophobic vesicle membrane.

23. The vesicle of claim 1 additionally comprising at least one PET agent compartmentalized within the hydrophobic vesicle membrane.

24. The vesicle of claim 1 additionally comprising at least one radiological imaging agent compartmentalized within the hydrophobic vesicle membrane.

25. The vesicle of claim 1 additionally comprising at least one PDT agent compartmentalized within the hydrophobic vesicle membrane.

26. The vesicle of claim 10 additionally comprising at least one of a secondary emissive agent, a cytotoxic agent, a magnetic resonance imaging (MRI) agent, positron emission tomography (PET) agent, radiological imaging agent or a photodynamic therapy (PDT) agent compartmentalized within the hydrophobic vesicle membrane.

27. The vesicle of claim 1 additionally comprising at least one of an emissive agent, a cytotoxic agent, a magnetic resonance imaging (MRI) agent, positron emission tomography (PET) agent, photodynamic therapy (PDT) agent, radiological imaging agent, ferromagnetic agent, or ferrimagnetic agent, where said emitter or agent is compartmentalized within the aqueous polymersome interior.

28. The vesicle of claim 1 additionally comprising at least one of an emissive agent compartmentalized within the aqueous polymersome interior.

29. The vesicle of claim 1 additionally comprising at least one MRI agent compartmentalized within the aqueous polymersome interior.

30. The vesicle of claim 1 additionally comprising at least one PET agent compartmentalized within the aqueous polymersome interior.

31. The vesicle of claim 1 comprising at least one radiological imaging agent compartmentalized within the aqueous polymersome interior.

32. The vesicle of claim 1 additionally comprising at least one PDT agent compartmentalized within the aqueous polymersome interior.

33. The composition of claim 11 additionally comprising at least one of a secondary emissive agent, a cytotoxic agent, a magnetic resonance imaging (MRI) agent, positron emission tomography (PET) agent, photodynamic therapy (PDT) agent, radiological imaging agent, ferromagnetic agent, or ferrimagnetic agent, where said emitter or agent is compartmentalized within the aqueous polymersome interior.

34. A method for making bioresorbable polymersomes comprising:
coating a solution of a block copolymer consisting of polyethylene oxide and polycaprolactone, dissolved in a solvent, into a thin-film on a surface;
evaporating at least a portion of the solvent;
contacting the film, coated with the block copolymer, with an aqueous solution; and
heating the aqueous solution at temperature of at least about 50° C.

35. The method of claim 34 where the block copolymer is an amphilphilic multiblock copolymer consisting of discrete polyethylene oxide and polycaprolactone blocks.

36. The method of claim 34 where the block copolymer is an amphilphilic random copolymer consisting of a discrete polyethylene oxide block and a random hydrophobic polymer block in which there exists an oligocaprolactone component.

37. The method of claim 34 wherein the polyethylene oxide having a number average molecular weight from about 2.0 to about 3.8 kD, the block copolymer having a fraction of polyethylene oxide of from about 11 to 20 percent by weight.

38. The method of claim 34 where the fraction of polyethylene oxide of from about 11.8 to 18.8 percent by weight.

39. The method of claim 34 where the number average molecular weight of the polycarporlactone is from about 9.5 to about 22.2 kD.

40. The method of claim 34 where the molecular weight of the polyethylene oxide is about 2 kD and the molecular weight of the polycaprolactone is about 12 kD.

41. The method of claim 34 where solvent removal is under reduced pressure.

42. The method of claim 34 where the aqueous solution is 200 to 300 milliosmolar sucrose, 0.9 wt % NaCl (in water), or PBS.

43. The method of claim 34 where the solvent is chloroform, methylene chloride, tetrahydrofuran, ethanol, methanol, dioxane, or mixture thereof.

44. The method of claim 34 where the surface is a polytetrafluoroethylene (PTFE) or glass.

45. The method of claim 34 where the surface which is coated with a thin-film of the block copolymer is subjected to sonication, physical agitation, and/or electric field while in contact with the aqueous solution.

46. The method of claim 45 where the sonication is performed for at least 20 minutes.

47. The method of claim 45 further comprising freezing and then thawing the aqueous solution at least once.

48. The method of claim 45 where the aqueous solution is pressurized and passed through a supported membrane.
